(19) United States
(12) Patent Application Publication
(10) Pub. No.: US 2023/0255802 A1
(43) Pub. Date: Aug. 17, 2023

(54) METHOD FOR CONTROLLING AN ORTHOPEDIC DEVICE AND ORTHOPEDIC DEVICE

(71) Applicants: OTTOBOCK SE & CO. KGAA, Duderstadt (DE); UNIVERSITEIT TWENTE, Enschede (NL); AALBORG UNIVERSITY, Aalborg East (DK)

(72) Inventors: Jose GONZALEZ VARGAS, Duderstadt (DE); Eileen FRERK, Enschede (NL); Guillaume DURANDAU, Enschede (NL); Strahinja DOSEN, Aalborg East (DK)

(21) Appl. No.: 18/006,288

(22) PCT Filed: Jul. 21, 2020
(86) PCT No.: PCT/EP2020/070539

§ 371 Date: Jan. 20, 2023

(51) Int. Cl. A61F 2/72; A61N 1/36

(52) U.S. Cl. CPC A61F 2/72; A61N 1/36014; A61F 2002/6827

(57) ABSTRACT

The invention deals with a method for controlling an orthopedic device, the method comprising the following steps of: —Providing input signals, —Using said input signals as input variables of a musculoskeletal model, —Determining feedback signals using said musculoskeletal model, —Transmitting said feedback signals to said user of said orthopedic device.

The invention deals with a method for controlling an orthopedic device and a corresponding orthopedic device.

Orthopedic devices comprise orthoses, prostheses, exoskeletons and other supporting means that are adjusted and meant to support body parts of the user of the orthopedic device. The devices can support, secure or protect limbs, joints or muscles of the user. The devices can replace missing limbs or body parts or they can support healthy body parts, like arms, legs or the back of the user in order to protect the user from fatigue.

Orthopedic devices often comprise actuators, such as motors, torque generators, damping elements or artificial muscles that have to be controlled during the use of the respective device by the user. In order to do so, the user has to be able to tell the orthopedic device what he intends to do next or what he wants to do the orthotic device next. The orthopedic device thus needs a user interface so that the user can communicate with the orthopedic device. Many different ways of controlling these devices are known from prior art. It is known to detect parameters of the orthopedic device such as accelerations, velocities, relative and absolute positions as well as angular velocities, angular accelerations and momentums. Other methods use electromyographic, forcemyographic, mechanomyographic data or measurement data from electroencephalography in order to control the orthopedic device. These methods are sometimes also called tactile Myography or force Myography and use force sensors to control prosthetic devices such as prosthetic hands. With these methods it is clearly possible to control the orthopedic device.

From U.S. Pat. No. 9,221,177 B2 it is known to feed joint angles, joint torque, foot-ground contact data and measured data into a neuromuscular model in order to control a robotic leg. The model evaluates from the data what the user of the robotic leg intended the leg to do and calculates control signals for the robotic leg in order to mimic the intended motion. These control signals are then used to control the robotic leg. A similar idea is disclosed in U.S. Pat. No. 8,864,846 B1.

Most of these methods however do not include feedback mechanisms in order to feedback the position, velocity, acceleration or orientation of the orthopedic device to the user or to feedback characteristics of modeled muscles of the affected limb to the user. This lack of feedback forces the user to always look at the orthopedic device in order to be able to check, whether the orthopedic device does what the user intended the device to do. Without this indirect feedback the user has in these cases no chance to verify that his orthopedic device is working as intended which leads to a very insecure feeling as well as a low performance in general.

Thus, many attempts have been made in the prior art to overcome this shortcoming. US 2016/0331561 A1 proposes to give sensory feedback to amputees and wearers of orthopedic devices. A feedback interface is proposed which might include vibrational or electric stimulation of the skin or direct stimulation of the user's nerves via intraneural electrodes.

The main question is thus, what information can be used as a feedback to the user. WO 2005/051329 A1 proposes to use sensors that are arranged on the orthopedic device and to use the measurement data of at least one of these sensors as a feedback to the user. Such a sensor can for example be a pressure sensor that is positioned on the fingertip of an artificial hand. This pressure sensor can then measure the grasp force if for example the user of the artificial hand grasps something.

Compared to the natural feedback, a human receives from his limbs, legs, arms, hands and other body parts, the sensor feedback received from an artificial feedback system from the orthopedic device is very late. These long latencies to feedback sensor data from the orthopedic device are among the main reasons hampering the effectiveness of artificial somatosensory feedback systems. Due to the delay, the value of the feedback information is very limited for tasks of daily living.

Furthermore, the variables that are transmitted using artificial feedback described in the state of the art are already available to the user through other intrinsic feedback sources, for example by visual observation or listening to the device. Relying only on sensor information for feedback makes it impossible to provide feedback signals to the user related to characteristics not measurable with a sensor at the orthopedic device. Thus, no feedback can be provided with the systems described in the prior art related to how a missing limb would feel or about characteristics of the missing muscles, such as muscle length, while performing the desired movement. This information is received from a healthy limb via proprioception, for example from muscle spindles.

It is thus an object of the present invention to provide a method for controlling an orthopedic device and a corresponding orthopedic device that overcomes or at least reduces the disadvantages of the prior art.

It is thus proposed a method for controlling an orthopedic device, the method comprising the following steps of: Providing input signals, Using said input signals as input variables of a musculoskeletal model, Determining feedback signals using said musculoskeletal model and Transmitting said feedback signals to said user of said orthopedic device.

The input signals comprise information about the intended movement or action the user of said orthopedic device intends to perform. The intended movement of the user can be a certain action, like walking on a slope, climbing stairs, sitting down, raising an arm or any other movement the orthopedic device is capable of performing. If the orthopedic device does not replace a missing limb or body part but supports or protects an existing one, any movement the supported or protected body part is or was capable of, can be an intended movement. The input signals are then used as input variables of the musculoskeletal model. An intended action can be to grasp an object, if for example the orthopedic device is an artificial hand. It is possible but not necessary to identify the intended movement or action in order to use the input signals in the musculoskeletal model. It is sufficient to detect physical parameters like electric current or voltage, torques, forces or the like without interpreting them to identify an intention or an intended movement or action. However, it is possible to use these parameters for controlling the orthopedic device and to determine feedback signals that are to be transmitted to the user. It makes this special embodiment of the method very intuitive and easy to adapt and to learn for the user of the orthopedic device.

The musculoskeletal model models at least one limb, a joint or a body part of a human body. It models bones, muscles, tendons, and joints with corresponding degrees of freedom. In addition, it preferably models Golgi tendon organs, muscle spindles, muscle-tendon kinematics and kinetics. Modelling Golgi tendon organs and muscle spindles allows to provide their firing patterns as feedback to the user just like the human nervous system would do. Usually only the missing limb or a paretic limb is modelled but it is in certain cases advantageous to also model other limbs or body parts. These other limbs or body parts can be the counterpart of a missing limb or adjacent body parts. If for example a left leg is missing, it is preferable not only to model the left leg itself but also the corresponding right leg (the contralateral leg) and/or the hip and the torso of the user.

While the model itself usually is a standard model it is preferable to adjust and tune it to fit to the individual user as good as possible. The standard model comprises information about which bones, muscles, tendons and joints are to be modeled and what the corresponding degrees of freedom and movement are. Preferably, also information about which Golgi tendon organs and muscle spindles are to be modeled is included in the model. This is then preferably individualized by the user's anthropometry, comprising e.g. individual lengths and weights of certain body parts. It is particularly advantageous to also include mechanical limitations of existing limbs of the user. This is important when the user has a paretic limb, which often occurs with stroke patients.

The model is then used to determine feedback signals that can then be transmitted to the user of the orthopedic device. Since the input signals fed into the model comprise information about the intended movement or action the user of the orthopedic device intends to perform next, the model can be used to model parameters of the orthopedic device during this intended movement or action. These parameters include at least one of position, orientation, velocity, acceleration, angular velocity of the orthopedic device or at least a part thereof, and forces and momentum or muscle length, muscle contraction velocity and muscle and/or tendon forces and joint torques acting on the orthopedic device or at least a part thereof. Based on these parameters extracted from the musculoskeletal model, feedback signals are determined which are then transmitted to the user.

Since these feedback signals and the underlying parameters can be determined using the model and preferably the model only, no delay and latencies occur due to measurements or other sensoric data. If the movement of the orthopedic device or at least a part of the orthopedic device and the model are in temporal correspondence the feedback signals are transmitted to the user in real time. Temporal correspondence in this context means that the orthopedic device performs as exact as possible the movement and/or action that is modelled by the model.

The model comprises a number of mathematical equations which are to be used to mathematically describe motions and acting forces that can occur with the orthopedic device. The model, and thus these mathematical equations and the parameters used in the model are stored in an electronic data storage. An electronic data processing device, in particular a microprocessor is used to carry out the necessary calculations based on the mathematical equations of the model in order to “model” a body part, a movement or action of the orthopedic device or at least a part thereof. It is also used to determine the necessary parameters. The electronic data processing device is capable of accessing the data storage, in which the necessary data for the model and for carrying out the necessary calculations is stored.

In other words, the musculoskeletal model module contains a mathematical model that describes the musculoskeletal system specific to the user. The model approximates the muscle and tendon dynamics as well as the body structure kinematics, and it is adjusted to the user's anthropometry. For amputees it reproduces and approximates the missing limb, and the musculoskeletal dynamics and kinematics as well.

In order to be able to determine viable feedback signals the model preferably comprises information about the orthopedic device. This in particular means information about the possible movements, the actuators, joints and other elements of the orthopedic device as well as information about the possible forces that can be applied by the orthopedic device. The information about the orthopedic device preferably comprise what sensors are available that can measure parameters of the orthopedic device. For example, angle sensors, accelerometers, etc. can be used to provide information about the position and/or orientation and/or velocities and/or accelerations of the orthopedic device or at least a part thereof. This can then be used to update the state of the model and calculate the next steps. The basic model can usually remain unchanged, but the individualization can be adjusted and optimized, preferably by tuning model parameters. The tuning of model preferably can be done using optimization, statistical or machine learning methods to learn from the user while using the system. Preferably the limits of the orthopedic device are programed as constrains of the model. For example, if the joint can only rotate 10 degrees, then this is indicated in the model.

The sensory feedback signals computed by the musculoskeletal model can be conveyed back to the user using temporal or spatial encoding, the choice of which depends on the impairment level such as the amputation level, patient characteristics and psychometrics, as well as the demands on the practical application such as the compactness of the interface. The temporal encoding will translate the estimated somatosensory variables into a pulse train that is the feedback signal and whose parameters are adjusted online to mimic the estimated somatosensory information. In this way the magnitude of the muscle force can be communicated through the intensity and/or frequency of stimulation. This allows using one stimulator actuator to convey somatosensory information with high temporal and amplitude resolution. The spatial feedback will relate the feedback signals to different stimulator actuators placed at different points of the limbs, muscles or nerves, which will be actuated accordingly to the value of the feedback variable. As an example, each stimulator can be associated to a specific level of muscle force. This concept can of course be transferred to other form of electrodes, such as nerve cuff electrodes.

In a preferred embodiment providing input signals comprises detecting measurement data from the user of the orthopedic device and/or the orthopedic device using at least one measurement device. A measurement device for detecting measurement data from the user can comprise at least one of an electromyography (EMG) sensor, high-density EMG (HD-EMG), a forcemyography (FMG) sensor, Mechanomyography sensors (MMG), ultrasound (US) sensors, an electroencephalogram (EEG), video sensors such as a camera or Lidar (light detection and ranging) system and an inertial sensor. A measurement device for detecting measurement data from the orthopedic device can comprise at least one of an inertial sensor, acceleration sensor, force sensor, pressure sensor, a sensor for detecting an electrical current and a temperature sensor.

Preferably the detected measurement data is processed, in particular filtered, to provide input signals from the measurement data. This processing can also be called a conditioning of the measurement data, so that conditioned data is obtained. For EMG and HD-EMG data for example, it is possible and preferable to convert the raw data into an EMG envelop. This is done using low pass filtering of the signal (between 3 to 10 Hz). If forcemyography signals are used, one preferably applies a low pass filter to remove movement artifacts. These are the most straight forward sensing methods that could be used with the model since it gives information of muscle activation and can be mapped to specific muscles in the model.

If acceleration data from an acceleration sensor is used, preferably a filtering is applied to obtain the acceleration trend. Alternatively or additionally it is possible to integrate the measurement value over time to obtain a velocity trend or to just detect peaks in the measurement data and use these as input signals. In this mode the detected acceleration of the orthopedic device can be used to trigger a central pattern generator oscillator, which governs the model timing. If an input signal such as EEG or ultrasound is used, then more complex filtering is preferred to get more meaningful information.

Preferably the measurement data comprises myoelectric signals picked up from the skin and/or at least one muscle and/or the nerves of said user.

In a preferred embodiment the method further comprises the step of determining control signals for said orthopedic device using said musculoskeletal model. The conditioned measurement data describing the user intent, is used as input signals of the musculoskeletal model. This serves as an input to the model to approximate what the dynamics and kinematics of the model should be like at the present moment in time. Also, it predicts what the model should be like in the future. For example, it can approximate what should be the joint stiffness at the moment for the phantom limb, and how the joint stiffness will change in the future. The model can also be used to approximate and predict other variables such as muscle length, joint acceleration, joint velocities, joint torque, joint damping, force and contraction velocity, firing of muscle spindles, firing of Golgi tendon organs. This calculation that is carried out in order to obtain these predictions is then converted into control signals, that are also called commands in the following, that can be used to control the orthopedic device to do what is needed at this point and at future points in time. These commands take into account the dynamics of the orthopedic device, since they were determined using the musculoskeletal model which comprises these parameters and information. Therefore, the dynamics of the device are preferable to be available within the musculoskeletal model as well. The control signal can be, for example, a torque signal or a current signal for an electric motor, it could also be a position signal for a hydraulic valve.

As already described the approximations and predictions obtained by the calculations using the musculoskeletal model are preferably also used as physiological information that is sent to the sensory feedback interface, in order to transmit feedback signals to the user. For example, this information are estimates of what the internal state of the phantom limb or a paretic limb should be concerning e.g. fiber-length, stiffness, damping, velocity, force, torque of joints and other parameters.

In this embodiment the musculoskeletal model is used for both determining the feedback signals and for determining the control signals. It is thus possible to achieve a very good temporal alignment between the movement and actions of the orthopedic device and the feedback signals that are transmitted to the user. Thus, in a preferred embodiment of the present invention the feedback signals and the control signals are determined using the same musculoskeletal model. By this an internally consistent system is achieved which improves the control of the orthopedic device. Because control signals and feedback signals originate from the same model, the control and feedback work especially good together, providing effective closed-loop control. Feedback and control are inherently coupled, good feedback cannot be exploited if the control is poor and vice versa. Furthermore, if the control and the feedback are too different they cannot provide satisfying results. Receiving feedback and control from the same model makes the signals internally consistent. The feedback will at least nearly perfectly reflect the control and vice versa and the orthopedic device feels more natural to the user

Preferably at least two different control signals for said orthopedic device are determined, wherein the at least two different control signals preferably are determined simultaneously. This allows for example to control the damping of a prosthetic knee and the torque of a prosthetic ankle at the same time with the same model. Furthermore, it enables the control of variable stiffness actuators, which possess at least two motors, one for controlling joint force and one for controlling the change of the stiffness of the actuators. It becomes possible to control two actuators or damping elements of the orthopedic device at the same time.

Preferably the feedback signals are somatosensory signals, that are transmitted to said user via at least one of electrotactile stimulators, vibrotactile stimulators, auditory stimulators, visual stimulators, mechanical stimulators capable of generating a force and/or a torque, cuff electrodes, temperature stimulators, subdermal electrodes, percutaneous electrodes, implanted electrodes, peripheral nerve electrodes or intramuscular electrodes. Preferably, the feedback signals are vibrations or surface electrical stimulation. Audio and visual feedback can be used for training issues mainly. Independent from the chosen kind of signal, the user has to learn to interpret the feedback signals and create a mental map between what they feel and the meaning of it. Hence, it is preferable to use a feedback scheme that is intuitive and easy to learn. For example, grasp force can be transmitted as vibration intensity. In this case, when the user feels a stronger vibration, then they can interpret this as a stronger grip. Peripheral electrodes include inter alia intra-fascicular electrodes, inter-fascicular electrodes and regenerative electrodes.

In a preferred embodiment of the method the feedback signals are determined using also sensor information provided by at least one sensor. The sensor information comprises information about at least one of the positions, the orientation, the velocity, the acceleration of said orthopedic device and/or a part thereof. It can additionally or as an alternative comprise information about at least one of a torque, a force and/or a momentum acting on said orthopedic device and/or a part thereof and/or information about the environment, such as temperature, surface, terrain information like texture or slope, or information about another body part of the user. By using also this sensor information in addition to the musculoskeletal model it becomes possible to check whether the expected and intended movement and/or action of the orthopedic device correspond and coincide with the movement and/or action the orthopedic device actually performs. By this the reliability of the feedback that the user receives via the transmitted feedback signals is increased by which also the trustworthiness increases. Furthermore, the sensor information can provide additional characteristics that cannot be assessed with the model such as the temperature of a touched object. Preferably, some of the sensor information are thus complementary to the information provided by the model.

Preferably the sensor information is used to inform, update, correct and/or amend said musculoskeletal model. This is particularly advantageous if the expected and intended movement and/or action of the orthopedic device does not or at least not fully correspond and coincide with the movement and/or action the orthopedic device actually performs. Then the measured sensor information is used to adjust and optimize the parameters of the musculoskeletal model.

If, for example, a joint of a limb of the user can only rotate 10 degrees, then this is indicated in the musculoskeletal model. If over time the conditions of the user change, e.g. due to a healing process, this can be recognized by the at least one sensor. If in this case the at least one sensor of the orthopedic device detects a larger angle at the joint, the model can then be adjusted by amending this constrains to allow for more movement. This is particularly important for orthoses, since rehabilitation is to be expected and changes in the limb characteristics is desirable.

If the at least one sensor of the orthopedic device indicates a mechanical state of the orthopedic device (e.g. grasp force, joint angle, joint acceleration, joint velocity, joint electric torque, object recognition, etc.) that differs from the one expected from the calculations carried out by the electronic data processing device using the model, this information can be used for different purposes. It is used as a kind of reset for the current state of the orthopedic device (e.g. joint position), which is used as an input to calculate the next step in time. It can also be used to update the model parameters. Since the sensor information provided by the at least one sensor might be delayed compared to the model predictions, the sensor information is used as a way to compare to past predictions and update the model parameters to improve the new predictions and approximations accordingly. This guarantees that the musculoskeletal model is grounded and “learns and optimize” the dynamic and kinematic characteristics of the model. This will make the models more precise, more user-specific, and reduce possible drift or errors.

The variables estimated by the electronic data processing device using the musculoskeletal model can be used standalone or together with the sensor information obtained from the sensors in the orthopedic device. The electronic data processing device preferably comprises a module that automatically determines which source of information is more meaningful at a given time.

All computations are preferably performed in a continuous cycle that updates the orthopedic device feedforward control and somatosensory feedback. This way the user can activate the orthopedic device and quickly determine if the orthopedic device's control algorithm is acting as expected. This can help the user adjust the control if deemed necessary without having to wait until the sensors of the orthopedic device transmit relevant changes in its state or until the desired movement is completed.

In a preferred embodiment said musculoskeletal model is capable of modelling muscle forces, joint torques, joint stiffnesses and/or joint dampings said user intends to exert by said input signals.

In a preferred embodiment the feedback signals are determined based on said muscle forces, joint torques, joint stiffnesses and/or joint dampings. In addition that said muscle forces, joint torques, joint stiffnesses and/or joint dampings can be encoded in the feedback signals. This means, that in these embodiments the user of the orthopedic device receives information about the position, velocity, force, pressure and the like of the human muscles, joints or other parts of the human body, instead of or additional to information about the orthopedic device. This is particularly interesting for users of prosthetic devices because the information they receive from the feedback signals correspond to the missing limb, that is replaced by the prosthetic device. This can lead to a more natural feeling of the prosthetic device and thus to a higher comfort.

Alternatively or additionally also the characteristics of the modeled bones, muscles, tendons and joints, for example of the missing limb, can be used as the feedback signal.

In a preferred embodiment, the model determines how muscle length, muscle contraction velocity and muscle and/or tendon forces of the limb would be for the desired movement and provide this as a feedback to the user. Also modeled firing patterns of Golgi tendon organs or muscle spindles can be used as a feedback reflecting very closely the signals which the nervous system of a healthy individual would receive. The orthopedic device would in this case perform the desired movement and the user would get a feedback signal closer related to how a healthy limb would feel. This can be especially helpful to reduce phantom pain.

These signals, being internal to the model, cannot be determined by the user through the intrinsic sources of feedback such as by listening to or looking at the device. Therefore, the artificial feedback will provide information that is otherwise unavailable to the user, hence facilitating the control of the device.

The invention also relates to an orthopedic device comprising an electronic controlling device capable of performing a method according to at least one embodiment of the present invention. The orthopedic device comprises an electronic data processing device. This is capable of accessing a data storage in which the mathematical equations and parameters for the model are stored. The data processing device preferably comprises an input sensor interface unit in which measurement data from the user is transformed into input variables for the musculoskeletal model. The data processing device preferably further comprises a model unit which is capable of handling the musculoskeletal model in order to calculate parameters and other characteristics needed for the methods according to embodiments of the present invention from the model.

A further model of the electronic data processing device is a feedback interface unit, in which feedback signals are determined from parameters and characteristics extracted and calculated from the model in the model unit. The feedback interface unit is capable of transforming these into feedback signals.

The model unit sends the needed information to the feedback interface unit. It can also send control signals that have be determined by the model unit to actuators of the orthopedic device which are controlled using these control signals.

Using the attached drawings different embodiments of the present invention are described in the following.

FIGS. 1-4 show flow diagrams of different methods according to different embodiments of the present invention.

FIG. 1 shows a pretty simple embodiment of a flowchart for a method for controlling an orthopedic device 2. First input signal have to be provided. According to method illustrated in FIG. 1 measurement data 8 is detected form a user 4, which are then processed in an input sensor interface 6. In this input sensor interface 6 the measurement data 8 detected by at least one sensor are transformed and translated into input variables 10 for a musculoskeletal model 12. This model 12 is used to determine control signals 14 for the orthopedic device 2 and to determine feedback signals 16 which are then after being processed in a sensory feedback interface 18 transmitted to the user 4. The orthopedic device is provided with at least one sensor, which is not shown in FIG. 1. This at least one sensor senses sensor information 20 which is used both to update and check the validity of the model 12 and to improve feedback in the sensory feedback interface 18. In the embodiment shown in FIG. 1 the input sensor interface, the musculoskeletal model 12 and the sensory feedback interface 18 are parts of an electronic data processing unit 22, illustrated by the dashed line.

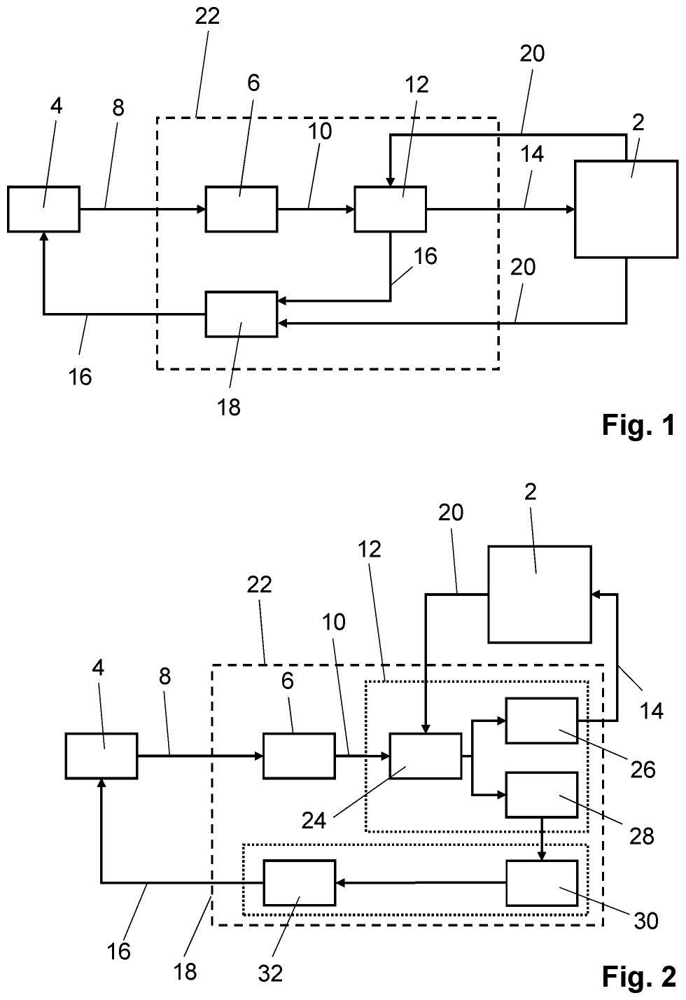

FIG. 2 is a more detailed flow chart. Again measurement data 8 are detected from the user 4 using at least one sensor. Said measurement data 8 is fed into the electronic data processing unit 22, which is again illustrated by the dashed line. The measurement data 8 is processed in the input sensor interface 6 which in this case is an EMG sensor interface, such as an 8 channel EMG sensor interface. When sensors other than an EMG sensor setup are used, also another input sensor interface 6 is to be used in order to be able to process forcemyography, ultrasound sensor or inertial sensor measurement data 8. The input variables 10 might for example be normalized EMG data based on the maximum voluntary contraction of the user 4. They are fed into the musculoskeletal model 12, which in FIG. 2 comprises several units and is thus illustrated by the dotted line. In the user-specific model unit 24 the input signals 10 are processed in order to determine an intended motion or action the user 4 intends to perform next. From this different movements, actions, tensions and/or forces of different elements of the orthopedic device are calculated in the user-specific model unit 24. These elements then are transformed in the device control transformation unit 26, which generates the control signals 14 which are then sent to the orthopedic device 2. The device 2 comprises at least one sensor the sensor information 20 of which is in the embodiment shown in FIG. 2 used only for updating the musculoskeletal model 24 in the user-specific model unit. The sensor information 20 can additionally or alternatively be used to update other units of the musculoskeletal model 12 and/or to improve the feedback signals 16 transmitted to the user 4.

In addition the elements calculated in the user-specific model 24 are also fed into a feedback normalization unit 28, which is also part of the musculoskeletal model 22. This unit 28 inter alia normalizes the joint moments that are calculated in the user-specific model unit 24 to the maximum closure joint moment that the user 4 can achieve as calculated by the user-specific model unit 24. This allows to measure the joint moments in percentages of the maximum joint moment. This of course is also done with other parameters that are calculated in the user-specific model unit 24 and that are to be used for the feedback that is to be transmitted to the user 4. The normalized parameters are then transformed into signal patterns in a feedback transformation unit 30 before they are transformed into feedback signals 16 in a feedback signal generator 32. These feedback signals 16 can be vibrotactile or other signals. The pattern generated in the feedback transformation unit 30 can be an intensity varying pattern or a temporal pattern. Of course, other patterns using other varying parameters can also be used. The feedback signal generator 32 and the feedback transformation unit 30 are two different parts of the sensory feedback interface 18 denoted by the dotted line.

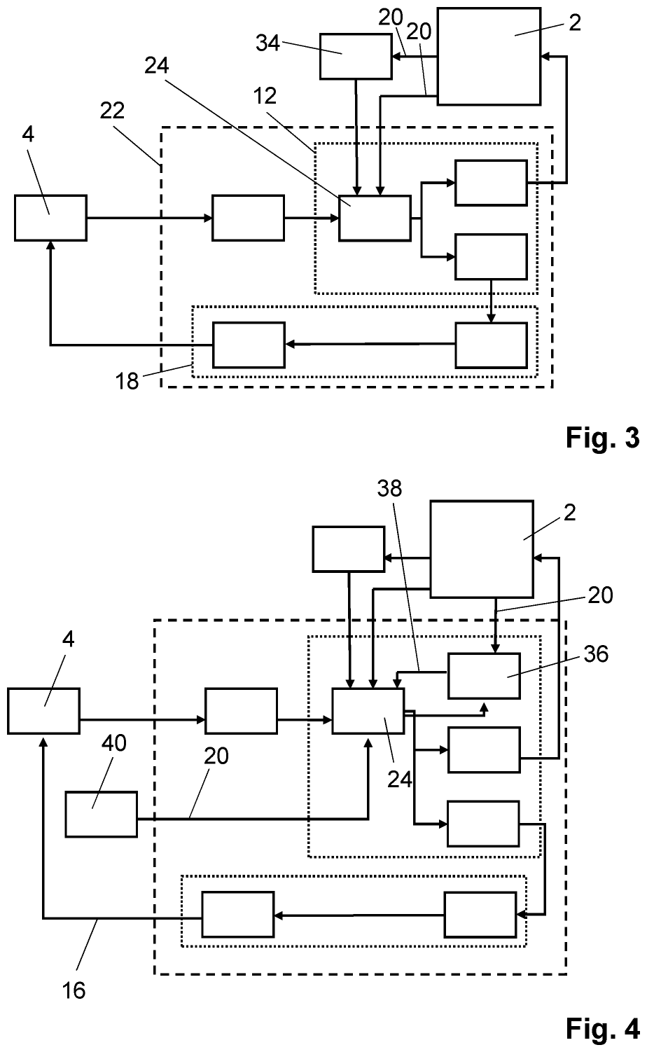

FIG. 3 shows a method very similar to the one of FIG. 2. It is used to control a prosthetic device for the lower limb, such as a leg prosthesis. The main difference to the more generically usable method of FIG. 2 is a gait cycle calculation unit 34. It is provided with sensor information 20 of at least one sensor positioned at or near the prosthetic device 2. This sensor information 20 is used to update and check the validity of the user-specific model unit 24 and the musculoskeletal model 12, but also to calculate when in a gait cycle the user 4 is. The sensor information 20 can be provided by an angular position sensor, an acceleration sensor and/or a gyroscope which are possibly available in the prosthetic device. The sensory feedback interface 18 and the electronic data processing unit 22 as well as the musculoskeletal model 12 remain the same as described with respect to FIG. 2.

FIG. 4 shows a method similar to the one in FIG. 3. The main difference is a parameter adjustment unit 36. This unit 36 is part of the musculoskeletal model 12 and the electronics data processing unit 22 and receives both sensor information 20 from at least one sensor of the orthopedic device 2 and elements and parameters calculated in the user-specific model unit 24. The parameter adjustment unit 36 compares expected values for different parameters that have been calculated in unit 24 with sensor information 20 from the at least one sensor on or at the orthopedic device 2. If deviations occur, that exceed a certain predetermined threshold, parameter information 38 is sent to the user-specific model unit 24 and the corresponding parameters become adjusted.

Another difference of the method according to FIG. 4 from the method according to FIG. 3 is the use of at least one sensor 40, that determines sensor information 20 that is basically or fully unrelated to the user and to the orthopedic device. This sensor information 40 is related to the environment the user 4 is in, such as weather conditions, temperature and moisture, and/or information concerning the ground the user 4 is walking on. This can relate to the slope and/or the kind of ground covering, such as grass, stone, wood or other materials. For the application in the upper limb orthopedic device, this information can be a snapshot of an object that is supposed to be grasped by a grasping device. This information also enters the musculoskeletal model 12 and is used to further improve the controlling of the orthopedic device 2 and the feedback signals 16 that are transmitted to the user 4.

### LIST OF REFERENCE NUMBERS

2 orthopedic device
4 user
6 input sensor interface
8 measurement data
10 input variables
12 musculoskeletal model
14 control signals
16 feedback signals
18 sensory feedback interface
20 sensor information
22 electronic data processing unit
24 user-specific model unit
26 device control transformation unit
28 feedback normalization unit
30 feedback transformation unit
32 feedback signal generator
34 gait cycle calculation unit
36 parameter adjustment unit
38 parameter information
40 sensor

1. A method for controlling an orthopedic device, comprising:
providing input signals,
using said input signals as input variables of a musculoskeletal model,
determining feedback signals using said musculoskeletal model,
transmitting said feedback signals to a user of said orthopedic device.

2. The method according to claim 1, wherein the step of providing input signals comprises detecting measurement data from the user of the orthopedic device and/or the orthopedic device using at least one measurement device.

3. The method according to claim 2, wherein the detected measurement data is processed to provide input signals from the measurement data of the at least one measurement device.

4. The method according to claim 2, wherein the measurement data comprise myoelectric signals picked up from skin and/or at least one muscle and/or nerves of said user.

5. The method according to claim 1 further comprising determining control signals for said orthopedic device using the musculoskeletal model or another musculoskeletal model.

6. The method according to claim 5, wherein the feedback signals and the control signals are determined using the same musculoskeletal model.

7. The method according to claim 5 wherein, at least two different control signals for said orthopedic device are determined in the determining control signals step.

8. The method according to claim 1 wherein the feedback signals are somatosensory signals, and further comprising transmitting the somatosensory signals to said user via at least one of electrotactile stimulators, vibrotactile stimulators, auditory stimulators, visual stimulators, mechanical stimulators capable of generating a force and/or a torque, cuff electrodes, temperature stimulators, subdermal electrodes, percutaneous electrodes, implanted electrodes, peripheral nerve electrodes or intramuscular electrodes.

9. The method according to claim 1 wherein the feedback signals are determined using sensor information provided by at least one sensor, wherein the sensor information comprise information about at least one of
position, orientation, velocity, and acceleration of said orthopedic device and/or a part thereof,
a torque, a force and/or a momentum acting on said orthopedic device and/or a part thereof,
environment of the orthopedic device, and
another body part of the user.

10. The method according to claim 7 further comprising updating, correcting or amending said musculoskeletal model using the sensor information.

11. The method according to claim 1 further comprising using said musculoskeletal model to model muscle forces, joint torques, joint stiffnesses and/or joint dampings said user intends to exert by said input signals.

12. The method according to claim 11, wherein the feedback signals are determined based on said muscle forces, joint torques, joint stiffnesses and/or joint dampings.

13. The method according to claim 11 wherein said muscle forces, joint torques, joint stiffnesses and/or joint dampings are encoded in the feedback signals.

14. An orthopedic device comprising an electronic controlling device which performs a method according to claim 1.

15. The method according to claim 7 wherein the at least two different control signals are determined simultaneously.

16. The method of claim 9 wherein the environment is selected from temperature, surface, and terrain.
